(19) United States
(12) Patent Application Publication
(10) Pub. No.: US 2017/0106027 A1
(43) Pub. Date: Apr. 20, 2017

(54) COMPOSITIONS AND METHODS OF IMPROVING PERFORMANCE AND INTESTINAL HEALTH OF POULTRY

(71) Applicant: Kemin Industries, Inc., Des Moines, IA (US)

(72) Inventors: Rachel Tonda, West Des Moines, IA (US); Jon Kieran Rubach, West Des Moines, IA (US); Sally Moore, Ankeny, IA (US); Mitchell Poss, Johnston, IA (US)

(21) Appl. No.: 15/295,753

(22) Filed: Oct. 17, 2016

Related U.S. Application Data
(60) Provisional application No. 62/241,871, filed on Oct. 15, 2015.

(51) Int. Cl. A61K 35/742; A61K 31/7034; A61K 36/48; A61K 36/49; A61K 36/22

(52) U.S. Cl. CPC A61K 35/742; A61K 36/49; A61K 36/22; A61K 36/48; A61K 31/7034

(57) ABSTRACT

Gut health in poultry is improved by providing an efficacious amount of extracts of tannic acid via feed or via drinking water. The extracts of tannic acid are also effective when the poultry are infected with coccidiosis and/or necrotic enteritis. In alternative embodiments, an efficacious amount of Bacillus coagulans is also included.

### BACKGROUND OF THE INVENTION

The present invention relates generally to compositions and methods of improving performance and intestinal health of poultry and, more specifically, to the use of feeding a hydrolysable tannin extract (tannic acid extract) in combination with Bacillus coagulans to improve the performance and intestinal health of poultry, including a reduction in the adverse effects of diseases causing intestinal stress as compared with those animals not fed such compositions.

Optimal nutrient uptake by the gastrointestinal tract is critical for productivity and feed efficiency of poultry. Irritation of the intestinal epithelium can result from growing stress, diet, viral or pathogenic infections. These stressors damage the intestinal barrier resulting in malabsorption of nutrients, increased incidence of diarrhea, inflammation, and oxidative stress which are linked to alterations in intestinal structure and barrier permeability. Increased intestinal barrier permeability allows toxins, microbes, and other pathogens access to the body interior and represents an overall decrease in intestinal integrity. The symptoms of intestinal stress result in a redistribution of energy towards suppressing the intestinal challenge and repairing the gut, which in turn decreases nutrient uptake and reduces animal efficiency.

Poultry raised in commercial scale operations are commonly exposed to parasitic and microbial gastrointestinal infections which induce intestinal stress and negatively impact productivity. For example, necrotic enteritis has been estimated to affect up to 40% of poultry flocks in the United States. Clinical necrotic enteritis is observed by sudden increases in flock mortality of up to 50%. However, the major challenge for US producers is mitigating subclinical necrotic enteritis which is considered more devastating due to the economic impact of long-term reductions in broiler performance.

Necrotic enteritis is caused by Clostridium perfringens, a gram-positive anaerobic bacterium which inhabits the poultry intestine. Under normal conditions, the healthy microbiome of the intestine keeps C. perfringens and its toxins at a low level. However, changes in the intestinal microflora or damage to the intestinal epithelium can allow for overgrowth of C. perfringens, leading to enterotoxemia and necrotic enteritis. While humid conditions and diets high in animal byproducts or grains are associated with causing necrotic enteritis, prior exposure of poultry to coccidiosis is considered the primary source of intestinal stress causing necrotic enteritis.

The cause of coccidiosis in poultry is the protozoan parasite, Eimeria sp. Of the seven species of Eimeria that commonly infect poultry, E. acervulina, E. maxima, and E. tenella are considered the most pathogenic and most common causes of coccidiosis. These parasites invade the intestinal epithelium in a site-specific manner causing inflammation and necrosis from the upper intestinal tract to the ceca. Eimeria maxima infection is specifically correlated to overgrowth of C. perfringens and development of necrotic enteritis. The lifecycle of Eimeria in which poultry naturally shed low levels of coccidial oocysts in feces creates further challenges for controlling the disease. Eimeria oocysts are environmentally hardy and the build-up of oocysts overtime can result in carry-over of coccidiosis infections to future poultry flocks. Necrotic enteritis thus remains a problem in poultry production because in involves the overgrowth of a bacteria that occurs naturally in the intestines of chickens and can be triggered by coccidiosis, one of the most prevalent diseases in poultry.

Control methods for mitigating intestinal stress typically include use of medicated feeds and/or medicated water containing anticoccidial chemicals or ionophores which alleviate the intestinal stress caused by Eimeria, as well as directly inhibit overgrowth of C. perfringens and other pathogenic microbials in the intestine. However, reports on the development of bacterial resistance to these control methods and potential presence of antibiotic residues in human food continues to be a concern. Coccidiosis vaccines provide an additional method for coccidiosis management as vaccination can initiate development of immunity to Eimeria and reduce the carryover of coccidial challenges due to late oocyst shedding in future broiler flocks. Unfortunately, in some instances immunization with live Eimeria oocysts can result in mild coccidial infections in vaccinated broilers, resulting in lower weight gain and higher feed conversion than non-vaccinated birds.

A need exists for compositions and methods to improve performance and intestinal health of poultry, especially compositions and methods capable of reducing or ameliorating the negative performance impacts of intestinal stress. These compositions and methods should ideally provide synergistic effects against multiple causes of intestinal stress in order to improve animal efficiency.

### SUMMARY OF THE INVENTION

The present invention provides methods and compositions for improving performance in production animals via improved weight gain, enhanced feed efficiency, and improving intestinal structure and function in poultry and other animals. Specifically, the present invention relates to compositions of hydrolysable tannins, tannic acid, more preferably an extract of hydrolysable tannins (tannic acid extract (TAE)) in combination with a direct-fed microbial, Bacillus coagulans, which improve performance and intestinal health of poultry exposed to a gastrointestinal challenge.

In some embodiments, the present invention is a composition for improved performance and/or intestinal health of poultry exposed to a gastrointestinal challenge.

In some embodiments, the present invention is a composition of tannic acid extract alone or combined with Bacillus coagulans, and optionally a carrier.

In some embodiments, the present invention is a composition where the tannic acid is extracted from gall nuts or tara pods through a water based extraction, an extraction using a mixture of water and an alcohol or organic solvent, or an extraction using organic solvent. (Reference:The Tannins. A Monograph on the History, Preparation, Properties, Methods of Estimation, and uses of the Vegetable Astringents, with an Index to the Literature of the Subject. (1892) Henry Trimble, J. P. Lippincott Co., Philadelphia. p. 78-87).

In some embodiments, the present invention is a method of application through feed or through water in the herein examples.

In some embodiments, the composition is administered to an animal in feed in an amount effective to decrease the growth of pathogenic bacteria in the animal gut. Such pathogenic bacteria could include Clostridium perfringens, Clostridium difficile, Salmonella, and E. coli. The composition administered should additionally show efficacy to reduce the growth of pathogenic parasites in the animal gut such as those of the Eimeria species.

In some embodiments, the composition is administered to an animal in feed in an amount directed for improving intestinal health of poultry vaccinated for coccidiosis and given a secondary challenge with Eimeria. A preferred coccidiosis vaccine is Advent®, a live coccidiosis vaccine. Such a secondary challenge could include a mixed infection of E. acervulina, E. maxima, and E. tenella. The method of the present invention may be used to decrease the Eimeria lesions of broilers exposed to a mixed Eimeria infection. Relatedly, the method of the present invention may be used to decrease the amount of Eimeria shed in animal feces.

In some embodiments, the composition is administered to an animal in feed in an amount directed for improving performance of poultry vaccinated for coccidiosis and given a secondary challenge with Eimeria. A preferred coccidiosis vaccine is Advent®, a live coccidiosis vaccine. Such a secondary challenge could include a mixed infection of E. acervulina, E. maxima, and E. tenella. The method of the present invention may improve weight gain and/or improve feed conversion of coccidiosis vaccinated broilers challenged with Eimeria.

In some embodiments, the composition is administered to an animal in feed in an amount directed for improving performance of poultry challenged with C. perfringens. The method of the present invention may be used to increase weight gain and improve feed conversion of broilers challenged with C. perfringens.

In some embodiments, the composition is administered to an animal in feed in an amount directed for improving intestinal health of poultry challenged with C. perfringens. The method of the present invention may be used to decrease necrotic enteritis lesions of broilers challenged with C. perfringens.

In some embodiments, the composition is administered to an animal in feed in an amount directed for improving intestinal health of poultry challenged with C. perfringens. The method of the present invention may be used to decrease mortality of broilers challenged with C. perfringens.

In some embodiments, the composition is administered to an animal in water in an amount directed for improving intestinal health of poultry challenged with C. perfringens. The method of the present invention may be used to increase weight gain and improve feed conversion of broilers challenge with C. perfringens.

In some embodiments, the composition is administered to an animal in water in an amount directed for improving intestinal health of poultry challenged with C. perfringens. The method of the present invention may be used to decrease necrotic enteritis lesions of broilers challenge with C. perfringens.

In some embodiments, the composition is administered to an animal in water in an amount directed for improving intestinal health of poultry challenged with C. perfringens. The method of the present invention may be used to decrease mortality of broilers challenge with C. perfringens.

### BRIEF DESCRIPTION OF THE FIGURES

FIG. 1 is a chart of the effects of treatments on Eimeria tenella sporozoite viability using a MTT in vitro assay. Treatments were as follows: control (no treatment), positive control (salinomycin, 60 ppm), tannic acid extract, tannic acid, and Bacillus coagulans. Tannic acid extract, tannic acid, and B. coagulans cell free supernatant were each tested at two concentrations 50 and 100 ppm.

FIG. 2 is an image of an in vitro well diffusion assay illustrating inhibition of the growth of C. perfringens. Treatments were as follows: positive control (chloramphenicol), tannic acid extract (TAE), and tannic acid extract combined with Bacillus coagulans (TAE+BC).

FIG. 3 is a chart of the comparison of average lesion scores from the vaccinated broilers during the Eimeria challenge (d28-35). To visually separate the Eimeria challenge groups, the Treatments in (Trts In) group (treatments kept in the basal diet for d28-35) is shown in blue and Treatments Out (Trts Out) group (treatments removed from basal diet on d28-35) is shown in orange. Treatment 1—basal diet without anticoccidial, Treatment 2—basal diet+BioCox® (60 g/t), Treatment 3—basal diet+Robenz® (33 g/t), Treatment 4—basal diet+tannic acid extract, Treatment 5—basal diet+tannic acid extract+B. coagulans.

FIGS. 4A and 4B are charts of the comparison of vaccinated broiler total oocyst per gram (OPG) in feces during the Eimeria challenge (d28-35). OPG counts were conducted six days after the Eimeria challenge (d35). A. Total OPG B. Total OPG y-axis zoomed in view. To visually separate the Eimeria challenge groups, the Treatments in (Trts In) group (treatments kept in the basal diet for d28-35) is shown in blue and the Treatments Out (Trts Out) group (treatments removed from basal diet on d28-35) is shown in orange. Treatment 1—basal diet without anticoccidial, Treatment 2—basal diet+BioCox® (60 g/t), Treatment 3—basal diet+Robenz® (33 g/t), Treatment 4—basal diet+tannic acid extract, Treatment 5—basal diet+tannic acid extract+B. coagulans.

FIGS. 5A and 5B are charts of the effects of treatments on broiler A. average weight gain (WG), and B. feed conversion ratio (FCR) over the challenge period (d14-21). Treatment 1 birds were unchallenged. Birds in treatments 2-6 were challenged with C. perfringens on d19, 20, and 21. Treatment 1—basal diet without antibiotic, Treatment 2—basal diet without antibiotic, Treatment 3—basal diet+bacitracin methylene disalicylate (BMD, 50 g/t)), Treatment 4—basal diet+Tannic acid extract form 2, Treatment 5—basal diet+tannic acid extract form 1, Treatment 6—basal diet+tannic acid extract+B. coagulans.

FIGS. 6A and 6B are charts of the effects of treatments on broiler A. average weight gain (WG), and B. feed conversion ratio (FCR) over the first three weeks of the study (d0-21). Treatment 1 birds were unchallenged. Birds in treatments 2-6 were challenged with C. perfringens on d19, 20, and 21. Treatment 1—basal diet without antibiotic, Treatment 2—basal diet without antibiotic, Treatment 3—basal diet+bacitracin methylene disalicylate (BMD, 50 g/t)), Treatment 4—basal diet+Tannic acid extract form 2, Treatment 5—basal diet+tannic acid extract form 1, Treatment 6—basal diet+tannic acid extract+B. coagulans.

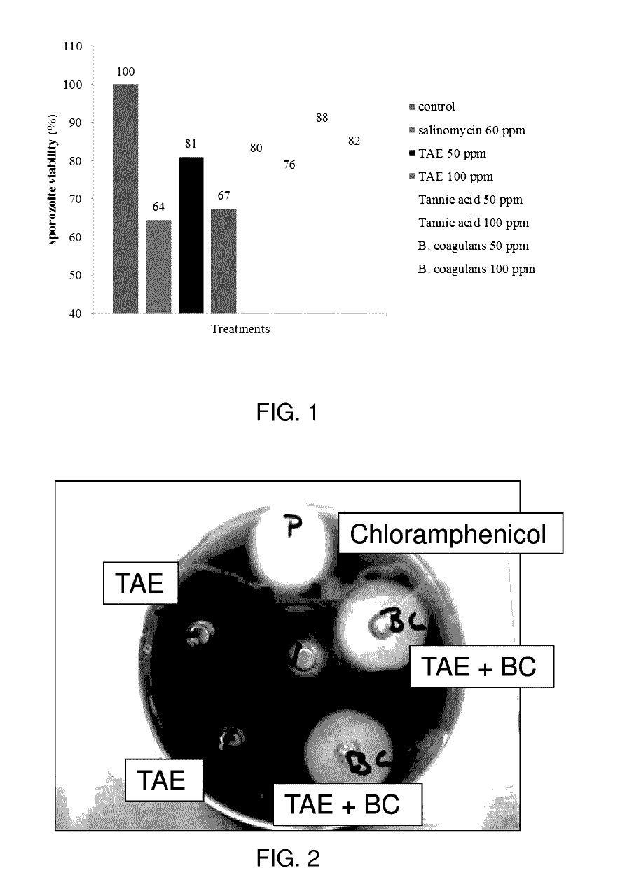

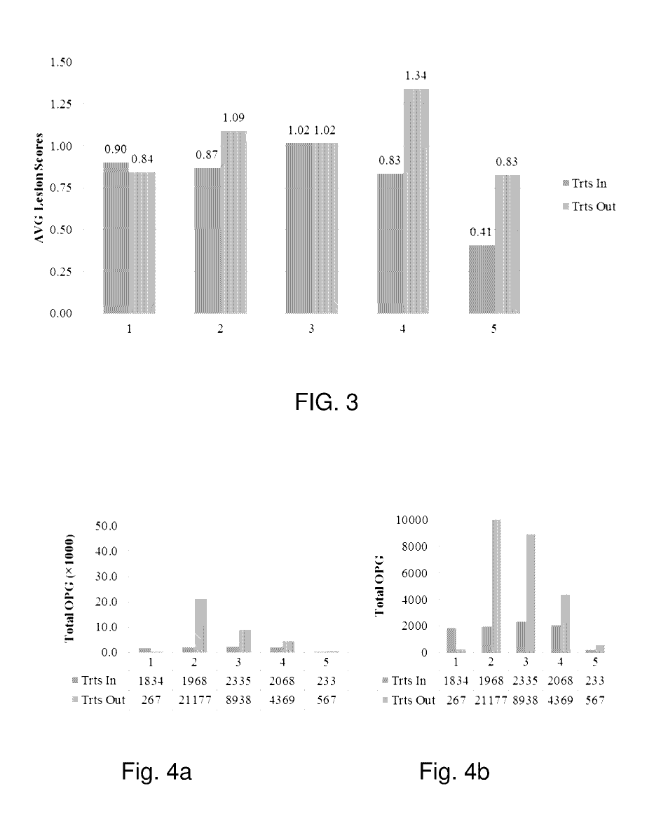

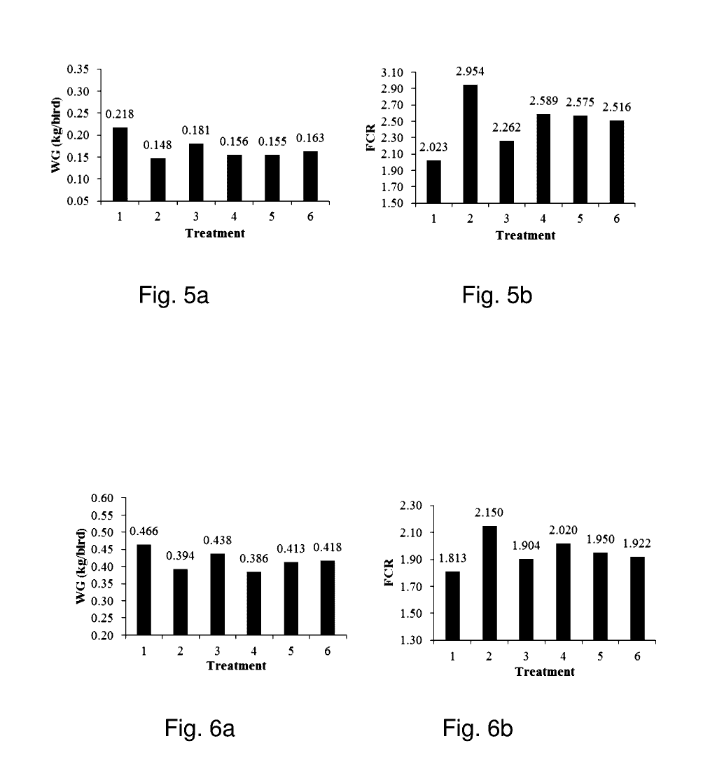

### DESCRIPTION OF THE INVENTION

### Example 1—Decreased Viability of Eimeria Sporozoites In Vitro with Tannic Acid Extract and Bacillus coagulans

An in vitro MTT assay was conducted to measure the effects of tannic acid extract, tannic acid, and Bacillus coagulans on the viability of Eimeria tenella sporozoites. Sporozoites were released from sporulated oocysts by the method described by Dulski et al. (Dulski, P., et al., The purification of sporocysts and sporozoites from Eimeria tanella oocysts using percoll density gradients; Avian Diseases, American Association of Avian Pathologists, Kennet Square, Pa., USA, vol. 32, no. 2, 1988). The sporozoites were sterilized, followed by incubation of the sporozoite suspension (minimum of 10⁵cells/mL) with 100 μL of tannic acid extract or 50 μL of B. coagulans cell free supernatant. This is followed by incubation of a MTT-PMS solution (0.2 millimolar each) with the sporozoite suspension (at 1:10 ratio) for 2 h at 41° C. After incubation, the contents were centrifuged at 800×g for 5 min and the supernatant was carefully removed. The purple colored formazan was dissolved in 200 μL DMSO and the absorbance was measured at 530 nm against a reference wavelength of 630 nm.

Sporozoite viability was reduced with all treatments, however reductions were greater with tannic acid extract and tannic acid compared to B. coagulans. Treatment with tannic acid extract at 100 ppm compared to 50 ppm decreased sporozoite viability by >10%, while increased dosage of tannic acid and B. coagulans only showed additional decreased viability of 5%. Tannic acid extract (100 ppm) reduced sporozoite viability equal to that observed with salinomycin.

### Example 2—Decreased Growth of Clostridium perfringens with Tannic Acid Extract and Bacillus coagulans In Vitro

An in vitro well diffusion assay was conducted to measure the ability of tannic acid extract and Bacillus coagulans to reduce the growth of C. perfringens. One gram of tannic acid extract or 1 g of tannic acid extract with B. coagulans concentrate (1×10⁸ spores) was dissolved with 9 mL of saline solution. The samples were vortexed, then each sample was diluted 1/10 in saline. The diluted samples were heat treated at 80° C. for 10 min then were placed in cold water and allowed to cool for 5 min. A 1 mL aliquot of the 1/10 dilution samples was added to 99 mL of Tryptic Soy Broth and placed in a shaking incubator at 37° C. for 24 hours. The cell free supernatants were prepared by centrifugation of the cultures at 5000×g for 10 min. Following centrifugation, the supernatants were filtered through 0.22μ filters. For the well diffusion assay, the Clostridium cultures were prepared by inoculating 0.1 mL from each seed stock into 9 mL of Cooked Meat Broth. The cultures were grown anaerobically over night at 37° C. To prepare the plates for the well diffusion assay, 1 mL of the overnight culture was dispensed into 99 mL of Reinforced Clostridial Medium (RCM) (Targeting 1×10⁶ CFU/mL) and gently swirled. The plates were poured and allowed to solidify. Once solidified, wells were aseptically cut into the agar. For the well diffusion assay, 100 μL of each cell free supernatant were placed into each of two separate wells.

The combination of tannic acid extract and Bacillus coagulans was found to inhibit growth of C. perfringens whereas tannic acid extract alone showed minimal growth inhibition. The zone of inhibition observed with the combination of tannic acid extract and Bacillus coagulans was similar to that observed with the positive control, chloramphenicol.

### Example 3—In Vivo Study of Tannic Acid Extract and Bacillus coagulans in Coccidiosis-Vaccinated Broilers

A 49-day trial with 3,000 Cobb×Cobb 500 male broiler chicks was conducted to investigate the effects of feed containing tannic acid extract and Bacillus coagulans on the performance and gut health of coccidiosis vaccinated broilers and vaccinated broilers challenged with a secondary Eimeria infection.

Day of hatch Cobb×Cobb 500 strain broiler chicks were obtained from Cobb-Vantress, Cleveland, Ga., USA. On day of hatch, prior to placement, all chicks were spray vaccinated with Advent®, a live coccidiosis vaccine, with the label recommended dosage via a Spraycox® machine. The Advent vaccine contains sporulated oocysts of E. acervulina, E. maxima, and E. tenella. To improve vaccine uptake, the chicks were allowed to preen for one hour prior to placement. Healthy appearing chicks were randomly selected from chick boxes and placed into 75 floor pens each containing 40 broiler chickens. Treatments were replicated in 15 blocks, randomized within blocks of five pens. Each floor pen had an average area of 50 ft² with built up wood shavings from three grow-out cycles as bedding, with a thickness of approximately four inches. The initial stocking density, after subtracting out for equipment, was 1.16 ft²/bird. Each pen had five feet high walls with the bottom one and a half feet being solid wood to prevent bird migration between pens.

A non-medicated (no antibiotic and no anticoccidial) corn-soybean based commercial type basal diet chicken ration was formulated with feedstuffs commonly used in the United States. The growth period was divided into three phases:starter (0-21 days), grower (21-35 days), and finisher (35-49 days). Birds selected for the study were fed the respective treatment diet in pelleted form with the starter phase provided in a crumbled pellet form. Treatment materials were added to the diet prior to pelleting, and all feed was pelleted at a set temperature of approximately 70° C. The diets and water were provided ad libitum throughout the experimental period. The diet composition and nutrient composition are outlined in Table 1 and Table 2, respectively.

TABLE 1
Composition of the basal diet used for all treatments in the
vaccination trial. Starter feed was fed as crumbled pellets
from 0-21 days. Grower feed was provided as pellets from d21-
35. Finisher feed was fed as pellets from d35-49.

| | Starter | Grower | Finisher |
|---|---|---|---|
| Ingredients | Feed (%) | Feed (%) | Feed (%) |
| Corn, yellow, grain | 55.44 | 60.05 | 65.37 |
| Soybean meal, | 35.71 | 31.10 | 26.38 |
| dehulled, solvent | | | |
| Corn DDGS | 4.00 | 4.00 | 4.00 |
| Fat, vegetable | 1.26 | 1.73 | 1.59 |
| Calcium carbonate | 1.15 | 1.11 | 0.92 |
| Dicalcium phosphate | 1.28 | 0.99 | 0.77 |
| Salt, plain (NaCl) | 0.44 | 0.42 | 0.43 |
| Methionine MHA | 0.35 | 0.26 | 0.22 |
| L-lysine | 0.21 | 0.20 | 0.17 |
| Trace Mineral | 0.08 | 0.08 | 0.08 |
| Vitamin Premix | 0.07 | 0.05 | 0.05 |
| Ronozyme P-(ct) | 0.02 | 0.02 | 0.02 |

TABLE 2
Nutrient composition of the basal diet used
for all treatments in the vaccination trial.

| | Starter | Grower | Finisher |
|---|---|---|---|
| | Feed | Feed | Feed |
| Nutrient | Amount (%) | Amount (%) | Amount (%) |
| Dry matter | 88.10 | 88.05 | 87.93 |
| Protein, crude | 22.93 | 21.01 | 19.12 |
| Fat, crude | 3.96 | 4.54 | 4.54 |
| Fiber, crude | 2.42 | 2.37 | 2.33 |
| Calcium | 0.92 | 0.83 | 0.70 |
| Phos. Total | 0.64 | 0.57 | 0.51 |
| Phos. Available | 0.45 | 0.40 | 0.36 |
| M.E. Poultry (kcal/kg) | 3000.00 | 3080.00 | 3130.00 |
| Methionine | 0.67 | 0.57 | 0.51 |
| Lysine | 1.42 | 1.28 | 1.13 |
| Tryptophan | 0.30 | 0.27 | 0.24 |
| Threonine | 0.93 | 0.85 | 0.77 |
| Sodium | 0.21 | 0.20 | 0.20 |
| Potassium | 0.89 | 0.81 | 0.74 |
| Chloride | 0.31 | 0.29 | 0.30 |
| Dig methionine | 0.63 | 0.53 | 0.48 |
| Dig cysteine | 0.31 | 0.29 | 0.27 |
| Dig lysine | 1.28 | 1.15 | 1.01 |
| Dig tryptophan | 0.29 | 0.26 | 0.23 |
| Dig threonine | 0.80 | 0.73 | 0.66 |
| Dig isoleucine | 1.04 | 0.94 | 0.84 |
| Dig leucine | 1.87 | 1.75 | 1.64 |
| Dig arginine | 1.41 | 1.27 | 1.14 |
| Dig phenylalanine | 1.14 | 1.04 | 0.95 |
| Dig TSAA* | 0.94 | 0.82 | 0.75 |

*TSAA = total sulfur amino acids

A total of five different treatments were tested in the study. All groups were vaccinated with Advent coccidiosis vaccine on day of hatch. The treatment groups used in the study were: 1) vaccinated control (basal diet with no treatment or feed additive); 2) BioCox® (basal diet treated with salinomycin (60 g/t)); 3) Robenz® (basal diet treated with Robenidine®, (30 g/t)); 4) Tannic acid extract (basal diet containing tannic acid extract) and 5) Tannic acid extract with Bacillus coagulans (basal diet containing tannic acid extract and Bacillus coagulans). A carrier and dust control agent were added to Treatments 4 and 5 with a 500 g/MT final dosage in the basal diet. The compositions percentages of Treatments 4 and 5 are shown in Table 3.

TABLE 3
Composition of Treatments 4 and 5. Treatments were prepared
with tannic acid extract (TAE) and Bacillus coagulans (BC).
Final treatment was applied at 500 g/MT in feed.*

| | | | Dust | | |
|---|---|---|---|---|---|
| Treatment | Description | Carrier (%) | Agent (%) | TAE (%) | BC (%) |
| 4 | TAE | 79.5 | 0.5 | 20.0 | — |
| 5 | TAE + BC | 70.5 | 0.5 | 20.0 | 9.0 |

*Treatments contained 100 ppm TAE and/or 1 × 10⁵ CFU/g in feed

The measured response variables included weights of birds and feed intake on d0, 21, 35, and 49. Means for pen feed consumption (FC), body weight gain (WG), average daily gain (ADG), feed conversion ratio (FCR), and mortality were measured. After d21, experimental blocks 12-15 were allocated to assess performance and gut health of vaccinated broilers exposed to a late Eimeria challenge. A total of four blocks, with four pens per treatment, were allocated for the growth performance and gut health of coccidiosis vaccinated broilers challenged with a secondary Eimeria infection portion of the trial. This subset of pens was further divided into two groups: Treatments In (blocks 12 and 13) and Treatments Out (blocks 14 and 15), resulting in two replicate pens per treatment in each group. On d28, birds and feed in blocks 12-15 were weighed and new feed was issued to each pen. Blocks 12 and 13 were provided with the same grower feed containing treatment materials as were provided for d0-28. In blocks 14 and 15, all pens were provided basal non-medicated not treated feed (Trt 1 diet) for the duration of the Eimeria challenge study. With this design, four replicate pens of the vaccinated not treated group (Treatment 1) were created, however the four replicates were considered separately during the blocking design of the challenge. On d29, all birds in blocks 12-15 were challenged with a mixed Eimeria challenge of sporulated oocysts. The coccidial inoculum was delivered in a 1.0 mL oral gavage and provided 100,000 E. acervulina oocysts, 50,000 E. maxima oocysts, and 75,000 E. tenella oocysts to each broiler.

Intestinal health response variables for the Eimeria challenged broilers were measured on d35. On d35 (6 days post infection), half of the birds from each pen (20 birds/pen) were sacrificed and lesion scored. The upper, middle, and cecal regions of the birds' intestines were scored for E. acervulina, E. maxima, and E. tenella, respectively, using the system of Johnson and Reid (Johnson, J, and Reid, W. M. (1970). Anticoccidial drugs: lesion scoring techniques in battery and floor-pen experiments with chickens. Experimental Parasitology 28: 30-36) wherein 0 is normal and 1, 2, 3, or 4 indicate increasing severity of infection. Individual as well as mean lesion scores for each pen were determined. Oocysts per gram (OPG) of Eimeria in feces were assessed on d35. Ten samples of feces were collected from each pen on d35 to determine oocyst shedding. A salt fecal floatation method (Long, P. L. (1970) Studies on the Viability of Sporozoites of Eimeria tenella. Z. Parasitenk, 35: 1-6) was utilized in which feces collected from each pen were pooled, thoroughly mixed, and OPG were microscopically counted for each sample using a McMaster counting chamber. The results of the secondary Eimeria challenge study are shown in Table 4 and 5.

TABLE 4
Average lesion scores for E. acervulina, E. maxima, E. tenella, and overall lesions of
vaccinated broilers challenged with Eimeria d28-35. Vaccinated, challenged birds were treated
with BioCox ®, Robenz ®, tannic acid extract (TAE), or TAE combined with B. coagulans
(TAE + BC). Challenged broilers were split into two groups in which treatment materials were
kept in the basal diet (Treatments In) or taken out of the basal diet (Treatments Out) during the
Eimeria challenge period, d28-35.

| | | Challenge | | | | |
|---|---|---|---|---|---|---|
| Treatment | Description | Group | E. acervulina | E. maxima | E. tenella | Overall |
| 1 | Not treated | Treatments In | 0.58 | 0.43 | 1.70 | 0.90 |
| 1 | Not treated | Treatments Out | 0.95 | 0.50 | 1.08 | 0.84 |
| 2 | BioCox ® | Treatments In | 0.95 | 0.60 | 1.05 | 0.87 |
| 2 | BioCox ® | Treatments Out | 1.50 | 0.78 | 1.00 | 1.09 |
| 3 | Robenz ® | Treatments In | 0.45 | 0.30 | 2.30 | 1.02 |
| 3 | Robenz ® | Treatments | 1.38 | 0.68 | 1.00 | 1.02 |
| | | Out | | | | |
| 4 | TAE | Treatments In | 0.75 | 0.30 | 1.45 | 0.83 |
| 4 | TAE | Treatments | 1.08 | 1.13 | 1.83 | 1.34 |
| | | Out | | | | |
| 5 | TAE + BC | Treatments In | 0.33 | 0.23 | 0.68 | 0.41 |
| 5 | TAE + BC | Treatments Out | 0.73 | 0.60 | 1.15 | 0.83 |

TABLE 5
Average oocysts per gram (OPG) in feces for E. acervulina, E. maxima, E. tenella, and
total OPG of vaccinated broilers challenged with Eimeria d28-35. Vaccinated, challenged
birds were treated with BioCox ®, Robenz ®, tannic acid extract (TAE),
or TAE combined with B. coagulans (TAE + BC). Challenged broilers were split
into two groups in which treatment materials were kept in the basal diet (Treatments In)
or taken out of the basal diet (Treatments Out) during the Eimeria challenge period, d28-35.

| | | Challenge | | | | Total |
|---|---|---|---|---|---|---|
| Treatment | Description | Group | E. acervulina | E. maxima | E. tenella | OPG |
| 1 | Not treated | Treatments In | 1034 | 33 | 767 | 1834 |
| 1 | Not treated | Treatments | 233 | 0 | 33 | 267 |
| | | Out | | | | |
| 2 | BioCox ® | Treatments In | 734 | 67 | 1167 | 1968 |
| 2 | BioCox ® | Treatments | 19510 | 0 | 1668 | 21177 |
| | | Out | | | | |
| 3 | Robenz ® | Treatments In | 1301 | 0 | 1034 | 2335 |
| 3 | Robenz ® | Treatments | 4869 | 600 | 3468 | 8938 |
| | | Out | | | | |
| 4 | TAE | Treatments In | 867 | 500 | 700 | 2068 |
| 4 | TAE | Treatments | 1668 | 900 | 1801 | 4369 |
| | | Out | | | | |
| 5 | TAE + BC | Treatments In | 67 | 0 | 167 | 233 |
| 5 | TAE + BC | Treatments | 267 | 0 | 300 | 567 |
| | | Out | | | | |

Data in tables 4 and 5 are the comparisons between the lesion scores and oocyst shedding of vaccinated broilers not treated, medicated with BioCox® or Robenz®, or fed tannic acid extract (TAE) with or without Bacillus coagulans (TAE+BC) during the secondary Eimeria challenge. Both lesion scores and OPG were commonly lower in the Treatments In group than in the Treatments Out group. Lesion scores showed minimal changes whether the treatment materials were kept in (Treatments In) or removed from (Treatments Out) the feed, whereas increased OPG in feces were observed when the treatment materials were removed from feed. Birds fed TAE+BC generally showed lower lesion scores and fewer oocysts in feces than birds fed TAE or birds medicated with BioCox® or Robenz®. The combination of TAE+BC additionally had lower E. acervulina, E. maxima, E. tenella, and overall lesion scores and OPG than TAE alone in both the Treatments In and Treatments Out groups. When treatments were kept in the feed, birds fed TAE+BC were the only group in which individual Eimeria lesion scores and OPG as well as average lesion scores and OPG were lower than the vaccinated control. When treatments were removed from birds, TAE+BC showed the least increase in total oocyst shedding compared to when the treatment materials were kept in the feed during the Eimeria challenge.

### Example 4—Efficacy of Tannic Acid Extract Formulations and Tannic Acid Extract with Bacillus coagulans to Reduce Necrotic Enteritis in Broilers Challenged with Clostridium perfringens

A 28 day study with 384 Cobb×Cobb 500 male broiler chicks was conducted to investigate the effects of tannic acid extract and Bacillus coagulans to suppress the negative performance and intestinal health consequences of a C. perfringens infection.

Day of hatch Cobb×Cobb 500 strain broiler chicks were obtained from Cobb-Vantress, Cleveland, Ga., USA. Healthy appearing chicks were randomly selected from chick boxes and placed into 48 cages of 8 broiler chickens. Treatments were replicated in 8 blocks, randomized within blocks of six cages. The experiments were conducted in Petersime battery cages with floor space of 0.63 sq. ft/bird where the cage served as the experimental unit. Uniform temperature of approximately 80° F. (26.7° C.) was maintained for the duration of the trial. A non-medicated (no antibiotic growth promoter and no anticoccidial drug) corn-soybean based mash starter ration was feed during the study. The formulated diet used was the same as described in Table 6 and 7.

TABLE 6
Composition of the non-medicated diet
used in the Eimeria challenge trial.

| | Application Rate | Percent |
|---|---|---|
| Ingredients | (lb/ton) | (%) |
| Corn, yellow, grain | 1142.14 | 57.107 |
| Soybean meal, dehulled, | 737.18 | 36.859 |
| solvent | | |
| Fat, vegetable | 46.74 | 2.337 |
| Calcium carbonate | 28.14 | 1.26 |
| Dicalcium phosphate | 25.20 | 1.407 |
| Salt, plain (NaCl) | 8.74 | 0.437 |
| Methionine MHA | 6.30 | 0.315 |
| L-lysine | 1.92 | 0.096 |
| Trace Mineral | 1.50 | 0.075 |
| Vitamin Premix | 1.30 | 0.065 |
| L-Threonine 98.5 | 0.46 | 0.023 |
| Ronozyme P-(ct) | 0.38 | 0.019 |

TABLE 7
Nutrient composition of the non-medicated diet
used in the Eimeria challenge trial.

| Nutrient | Amount (%) | Nutrient | Amount (%) |
|---|---|---|---|
| Dry matter | 88.08 | Dig methionine | 0.58 |
| Protein, crude | 23.44 | Dig cysteine | 0.32 |
| Fat, crude | 4.57 | Dig lysine | 1.2 |
| Fiber, crude | 2.38 | Dig tryptophan | 0.29 |
| Calcium | 0.9 | Dig threonine | 0.81 |
| Phos. Total | 0.6 | Dig isoleucine | 1.04 |
| Phos. Available | 0.42 | Dig histidine | 0.57 |
| M.E. Poultry (kcal/kg) | 3,067 | Dig valine | 1.14 |
| Methionine | 0.62 | Dig leucine | 1.89 |
| Lysine | 1.35 | Dig arginine | 1.45 |
| Tryptophan | 0.3 | Dig phenylalanine | 1.12 |
| Threonine | 0.95 | Dig TSAA* | 0.9 |
| Sodium | 0.21 | | |
| Potassium | 0.84 | | |
| Chloride | 0.28 | | |

*TSAA = total sulfur amino acids

A total of six different treatments were tested in the challenge study. All treatment groups were challenged with 5,000 oocysts/bird of Eimeria maxima by oral gavage on d14. On d19, 20, and 21 all birds, except treatment 1, were orally inoculated with 1.0E8 CFU/mL of C. perfringens. A field isolate of C. perfringens known to cause necrotic enteritis and originating from a commercial broiler operation was utilized as the challenge organism. Fresh C. perfringens inoculum (1.0E8 CFU/mL) in a 1.0 mL oral gavage was provided to each bird on each of the three days. The treatment groups used in the study were: 1) unchallenged control (not treated, unchallenged); 2) challenged control (not treated, challenged); 3) Bacitracin methylene disalicylate (BMD) medicated, challenged (50 g/t); 4) Coated tannic acid extract treated, challenged (200 g/MT); 5) Tannic acid extract treated, challenged (200 g/MT) and 6) Tannic acid extract combined with Bacillus coagulans (1.0E5 CFU/g in feed) treated, challenged (200 g/MT). A carrier and dust control agent were added to Treatments 5 and 6 for a 200 g/MT final dosage in the feed. The compositions percentages of Treatments 5 and 6 are shown in Table 8.

TABLE 8
Inclusion levels of ingredients used in Treatments 5 and 6. Treatments
were prepared with tannic acid extract (TAE) and Bacillus coagulans
(BC). Final treatment was applied at 200 g/MT in feed.*

| | | | Dust | | |
|---|---|---|---|---|---|
| Treatment | Description | Carrier (%) | Agent (%) | TAE (%) | BC (%) |
| 5 | TAE | 54.0 | 0.5 | 45.5 | — |
| 6 | TAE + BC | 31.4 | 0.5 | 45.5 | 22.6 |

*Treatments contained 91 ppm TAE and/or 1 × 10 CBU/g in feed

The response variables measured included necrotic enteritis lesion scores, mortality, and performance. On d21, three birds from each cage were selected, sacrificed, weighed, and examined for the presence of necrotic enteritis lesions. Lesion scores were determined by using the necrotic enteritis lesion scoring system which was based on a 0 to 3 score, with 0 being no lesions, 1 being mild lesions, 2 being moderate lesions, and 3 being the marked to severe lesions (Hofacre, C. L., Beacom, T., Collett, S., and Mathis, G. (2003). Using competitive exclusion, mannan-oligosaccharide and other intestinal products to control necrotic enteritis. J. Appl. Poult. Res., 12: 60-64). Individual as well as mean lesion scores by cage were provided. The birds and feed were weighed by pen on d0, 14, 21, and 28. Means for cage weight gain (d0-14, 14-21, 0-21, and 0-28), feed consumption, and feed conversion ratio (FCR) were then calculated. FCR was adjusted to account for mortality occurring during the study. Results of for FCR for 0-21 days during the study are shown in Table 9.

TABLE 9
Effects of Tannic acid extract (TAE) and TAE combined with
Bacillus coagulans (TAE + BC) on feed conversion
ratio (FCR) of broilers challenged with C. perfringens.

| | | FCR (0-21 |
|---|---|---|
| TRT | Description | days) |
| 1 | Unchallenged control | 1.813 |
| 2 | Challenged control | 2.150 |
| 3 | BMD | 1.904 |
| 4 | Coated TAE | 2.020 |
| 5 | TAE | 1.951 |
| 6 | TAE + BC | 1.922 |

Differences in necrotic enteritis lesion scores were minimal among the C. perfringens challenged groups, but mortality due to necrotic enteritis was reduced in birds medicated with BMD or treated with TAE or TAE+BC. Administration of BMD, TAE, or TAE+BC improved FCR of C. perfringens challenged broilers. Of the TAE formulations assessed during the study, TAE+BC generally showed the highest weight gain, lowest FCR, and most similar performance to birds treated with BMD.

### Example 5—Effects of Tannic Acid Extract and Direct-fed Microbial Combinations to Control Clostridium perfringens Induced Necrotic Enteritis in Broilers

A 28 day study with 560 Cobb×Cobb 500 male broiler chicks was conducted to investigate the effects of tannic acid extract (TAE) and direct-fed microbial (DFM) formulations to increase resistance to C. perfringens induced necrotic enteritis.

Day old male Cobb×Cobb 500 broiler chicks were obtained from Cobb-Vantress, Cleveland, Ga., USA. Health appearing chicks were randomly selected from chick boxes and placed into 70 cages of 8 broiler chickens. Treatments were replicated in 10 blocks, randomized within blocks of seven cages. The experiments were conducted in Petersime battery cages with floor space of 0.63 ft²/bird where the cage served as the experimental unit. The feeder space per bird was 8 birds per 24×3.5 inch feeder. Uniform temperature of approximately 80° F. (26.7° C.) was maintained for the duration of the trial. A non-medicated (no antibiotic and no anti-coccidial) corn-soybean based pelleted diet starter ration was fed during the study. Treatment materials were added to the diet prior to pelleting, and all feed was pelleted at a set temperature of approximately 70° C. Treatment material provided through the water was added to fresh water on a daily basis. The diets and water were provided ad libitum throughout the experimental period. The diet composition and nutrient composition are outlined in Tables 10 and 11, respectively.

TABLE 10
Composition of the basal diet used in
the necrotic enteritis challenge trial.

| | Application Rate | Percent |
|---|---|---|
| Ingredients | (lb/ton) | (%) |
| Corn, yellow, grain | 1142.14 | 57.107 |
| Soybean meal, dehulled, | 737.18 | 36.859 |
| solvent | | |
| Fat, vegetable | 46.74 | 2.337 |
| Calcium carbonate | 28.14 | 1.26 |
| Dicalcium phosphate | 25.20 | 1.407 |
| Salt, plain (NaCl) | 8.74 | 0.437 |
| Methionine MHA | 6.30 | 0.315 |
| L-Lysine | 1.92 | 0.096 |
| Trace Mineral | 1.50 | 0.075 |
| Vitamin Premix | 1.30 | 0.065 |
| L-Threonine 98.5 | 0.46 | 0.023 |
| Ronozyme P-(ct) | 0.38 | 0.019 |

TABLE 11
Nutrient composition of the basal diet used
in the necrotic enteritis challenge trial.

| Nutrient | Amount (%) | Nutrient | Amount (%) |
|---|---|---|---|
| Dry matter | 88.08 | Dig methionine | 0.58 |
| Protein, crude | 23.44 | Dig cysteine | 0.32 |
| Fat, crude | 4.57 | Dig lysine | 1.2 |
| Fiber, crude | 2.38 | Dig tryptophan | 0.29 |
| Calcium | 0.9 | Dig threonine | 0.81 |
| Phosphorus Total | 0.6 | Dig isoleucine | 1.04 |
| Phosphorus Available | 0.42 | Dig histidine | 0.57 |
| Metabolizable Energy | 3,067 | Dig valine | 1.14 |
| (M.E.) Poultry (kcal/kg) | | | |
| Methionine | 0.62 | Dig leucine | 1.89 |
| Lysine | 1.35 | Dig arginine | 1.45 |
| Tryptophan | 0.3 | Dig phenylalanine | 1.12 |
| Threonine | 0.95 | Dig TSAA* | 0.9 |
| Sodium | 0.21 | | |
| Potassium | 0.84 | | |
| Chloride | 0.28 | | |

*TSAA = total sulfur amino acids

A total of seven different treatments were tested in the challenge study. All treatment groups were challenged with 5,000 oocysts/bird of Eimeria maxima by oral gavage on d13. On d18, 19, and 20 all birds, except treatment 1, were orally inoculated with 1.0E8 CFU/mL of C. perfringens. A field isolate of C. perfringens known to cause necrotic enteritis and originating from a commercial broiler operation was utilized as the challenge organism. Fresh C. perfringens inoculum (1.0E8 CFU/mL) in a 1.0 mL oral gavage was provided to each bird on each of the three days. The treatment groups used in the study were: 1) unchallenged control (not treated, unchallenged); 2) challenged control (not treated, challenged); 3) Bacillus subtilis (1.0E6 CFU/g in feed) treated, challenged (0.5 lb/t); 4) Tannic acid extract treated, challenged (0.5 lb/t); 5) Tannic acid extract combined with Bacillus coagulans (1.0E4 CFU/g in feed) treated, challenged (0.5 lb/t); 6) Tannic acid extract combined with Bacillus subtilis (1.0E6 CFU/g in feed) treated, challenged (0.5 lb/t); 7) Tannic acid extract combined with Bacillus coagulans (1.0E4 CFU/mL in drinking water) treated, challenged (0.335 g/L). Fresh treated drinking water was prepared on a daily basis. A carrier and dust control agent were added to Treatments 3-6 for a 0.5 lb/t final dosage. A carrier was added to Treatment 7 for a 0.335 g/L final dosage. The composition percentages of Treatments 3-6 and for Treatment 7 are shown in Tables 12 and 13, respectively.

TABLE 12
Composition of Treatments 3-6. Treatment were prepared with tannic
acid extract (TAE), Bacillus coagulans (BC), and Bacillus subtilis (BS).
Final treatment was applied at 0.5 lb/ton in feed.

| | | Carrier | Dust | TAE | | |
|---|---|---|---|---|---|---|
| Treatment | Description | (%) | Agent (%) | (%) | BC (%) | BS (%) |
| 3 | BS | 94.7 | 0.5 | — | — | 4.8 |
| 4 | TAE | 65.9 | 1.0 | 33.1 | — | — |
| 5 | TAE + BC | 65.3 | 1.0 | 33.1 | 0.6 | — |
| 6 | TAE + BS | 61.1 | 1.0 | 33.1 | — | 4.8 |

Treatments contained 91 ppm TAE and/or 1 × 10⁴ CFU/g BC and/or 1 × 10⁶ CFU/g BS in feed.

TABLE 13
Composition of water soluble (WS) Treatment 7. Treatment was
prepared with tannic acid extract (TAE) and Bacillus coagulans
(BC). Final treatment was applied at 0.335 g/L in water.

| Raw Material (%) | TAE + BC WS |
|---|---|
| TAE (%) | 22.5 |
| BC (%) | 0.5 |
| Carrier (%) | 77.0 |

Treatment contained 91 ppm TAE and 1 × 10⁴ CFU/mL BC in water.

The response variables measured included necrotic enteritis lesion scores, mortality, and performance. On d20, three birds from each cage were selected, sacrificed, weighed, and examined for the presence of necrotic enteritis lesions. Lesion scores were determined by using the necrotic enteritis lesion scoring system which was based on a 0 to 3 scale, with 0 being no lesions, 1 being mild lesions, 2 being moderate lesions, and 3 being marked to severe lesions (Hofacre, C. L., Beacom, T., Collett, S., and Mathis, G. (2003). Using competitive exclusion, mannan-oligosaccharide and other intestinal product to control necrotic enteritis. J. Appl. Poult. Res., 12: 60-64). Individual as well as mean lesion scores by cage were provided. Necrotic enteritis lesion scores and mortality were reduced with BS, TAE, TAE+BC, and TAE+BS compared to the challenged control (Table 14). Combinations of TAE with a Bacillus organism showed lower lesions than TAE when provided to birds through the feed. Of the challenged treatments, TAE+BC WS had the lowest mortality (7.5%) of the C. perfringens challenged groups.

TABLE 14
Effect of Tannic acid extract (TAE) and TAE combined with direct-
fed microbials Bacillus subtilis (BS) or Bacillus coagulans
(BC) on necrotic enteritis lesion scores and necrotic enteritis
mortality of broilers challenged with C. perfringens. A water
soluble formulation of TAE + BC (TAE + BC WS) was
provided to birds in TRT 7 via the drinking water.

| TRT | Description | Lesion Score | Mortality (%) |
|---|---|---|---|
| 1 | Unchallenged control | 0.1 | 0.0 |
| 2 | Challenged control | 2.1 | 33.8 |
| 3 | BS | 1.1 | 17.5 |
| 4 | TAE | 1.1 | 12.5 |
| 5 | TAE + BC | 0.8 | 13.8 |
| 6 | TAE + BS | 0.8 | 13.8 |
| 7 | TAE + BC WS | 1.2 | 7.5 |

The birds and feed were weighed by pen on d0, 13, 20, and 28. Means for cage weight gain (d0-13, 13-20, 13-28, 0-21, and 0-28), feed consumption, and feed conversion ratio (FCR) were then calculated. FCR was adjusted to account for mortality occurring during the study. Minimal differences in performance were observed during the pre-challenge period (d0-13). Performance results for d13-20, d13-28, and d0-20 and d0-28 are shown in Tables 15, 16, and 17, respectively.

TABLE 15
Effect of Tannic acid extract (TAE) and TAE combined with
direct-fed microbials Bacillus subtilis (BS)or Bacillus coagulans
(BC) on weight gain (WG) and feed conversion ratio (FCR) of
broilers challenged with C. perfringens. A water soluble
formulation of TAE + BC (TAE + BC WS) was provided
to birds in TRT 7 via the drinking water.

| | | WG (13-20 days) | |
|---|---|---|---|
| TRT | Description | (kg/bird) | FCR (13-20 days) |
| 1 | Unchallenged control | 0.153 | 1.941 |
| 2 | Challenged control | 0.117 | 2.351 |
| 3 | BS | 0.148 | 2.097 |
| 4 | TAE | 0.128 | 2.387 |
| 5 | TAE + BC | 0.145 | 2.160 |
| 6 | TAE + BS | 0.124 | 2.341 |
| 7 | TAE + BC WS | 0.125 | 2.237 |

TABLE 16
Effect of Tannic acid extract (TAE) and TAE combined with
direct-fed microbials Bacillus subtilis (BS) or Bacillus coagulans
(BC) on weight gain (WG) and feed conversion ratio (FCR) of
broilers challenged with C. perfringens. A water soluble
formulation of TAE + BC (TAE + BC WS) was provided
to birds in TRT 7 via the drinking water.

| | | WG (13-28 days) | |
|---|---|---|---|
| TRT | Description | (kg/bird) | FCR (13-28 days) |
| 1 | Unchallenged control | 0.505 | 1.664 |
| 2 | Challenged control | 0.305 | 2.462 |
| 3 | BS | 0.415 | 1.897 |
| 4 | TAE | 0.428 | 1.856 |
| 5 | TAE + BC | 0.441 | 1.928 |
| 6 | TAE + BS | 0.445 | 1.896 |
| 7 | TAE + BC WS | 0.485 | 1.758 |

TABLE 17
Effect of Tannic acid extract (TAE) and TAE combined with
direct-fed microbials Bacillus subtilis (BS) or Bacillus coagulans
(BC) on weight gain (WG) and feed conversion ratio (FCR) of
broilers challenged with C. perfringens. A water soluble
formulation of TAE + BC (TAE + BC WS) was provided
to birds in TRT 7 via the drinking water.

| | | WG (kg/bird) | | FCR | |
|---|---|---|---|---|---|
| TRT | Description | d 20 | d 28 | d 20 | d 28 |
| 1 | Unchallenged control | 0.338 | 0.691 | 1.822 | 1.694 |
| 2 | Challenged control | 0.301 | 0.490 | 2.066 | 2.141 |
| 3 | BS | 0.350 | 0.617 | 1.856 | 1.798 |
| 4 | TAE | 0.320 | 0.620 | 2.023 | 1.820 |
| 5 | TAE + BC | 0.336 | 0.631 | 1.910 | 1.823 |
| 6 | TAE + BS | 0.323 | 0.644 | 2.010 | 1.856 |
| 7 | TAE + BC WS | 0.314 | 0.665 | 2.044 | 1.815 |

Necrotic enteritis challenged broilers treated with TAE, DFM, or TAE+DFM combination were observed to have improved performance compared to unchallenged control birds throughout this study. Combination treatments, TAE+BS and TAE+BC resulted in numerically lower necrotic enteritis lesions than broilers fed individual ingredients, however performance improvements tended to be larger in the TAE+BC combination. Broilers treated with TAE+BC either via feed or via water typically showed improved weight gain and FCR compared to the challenged control and challenged birds fed TAE only. Throughout the study, TAE+BC was observed to provide similar performance and health improvement benefits to broilers whether applied via the feed or via the drinking water._The present study has shown that TAE, DFM, and combinations thereof, especially TAE+BC, can improve broiler resistance to necrotic enteritis.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

We claim:

1. A method of improving gut health in poultry or other animals, comprising feeding an efficacious amount of (a) hydrolyzable tannins, tannic acid or more preferably extract of hydrolyzable tannins (tannic acid extract), selected from the group consisting of Quercus infectoria, Rhus chinensis and Caesalpinia spinosa and (b) a probiotic of the Bacillus species.

2. The method of claim 1, wherein the animals are infected with coccidiosis.

3. The method of either claim 1 or claim 2, further comprising feeding an efficacious amount of a Bacillus spp. strain selected from the group consisting of Bacillus coagulans, Bacillus subtilis, Bacillus licheniformis, Bacillus lentus and Bacillus pumilis.

4. A method of improving the performance of poultry or other animals, comprising feeding (a) an efficacious amount of hydrolyzable tannins, tannic acid or more preferably extract of hydrolyzable tannins (tannic acid extract) and (b) a probiotic of the Bacillus species.

5. The method of claim 4, wherein the animals are infected with coccidiosis.

6. The method of either claim 3 or claim 4, further comprising feeding an efficacious amount of a Bacillus spp. strain selected from the group consisting of Bacillus coagulans, Bacillus subtilis, Bacillus licheniformis, Bacillus lentus and Bacillus pumilis.

7. A composition for controlling necrotic enteritis in animals, comprising hydrolysable tannins, tannic acid or more preferably extract of hydrolysable tannins (tannic acid extract) and a probiotic of the Bacillus species.

8. The composition of claim 7, wherein the extract of hydrolysable tannins is selected from the group consisting of Quercus infectoria, Rhus chinensis and Caesalpinia spinosa

9. The composition of claim 7, wherein the probiotic is a strain selected from the group consisting of Bacillus coagulans, Bacillus subtilis, Bacillus licheniformis, Bacillus lentus and Bacillus pumilis.

10. The composition of claim 7, wherein controlling necrotic enteritis is selected from the group consisting of reduction in necrotic enteritis lesions, reduction in Eimeria lesions, reduction in Eimeria oocysts per gram of fecal matter and mortality in poultry.

11. The composition of claim 7 added to an animal feed or to animal drinking water, wherein the concentration of tannic acid in animal feed is between 25 ppm and 500 ppm.

12. The composition of claim 9 added to an animal feed or to animal drinking water, wherein the strain is Bacillus coagulans and the concentration of Bacillus coagulans in the animal feed is between 1×10² and 1×10⁶ CFU/g of said animal feed.

13. The composition of claim 9 added to an animal feed or to animal drinking water, wherein the strain is Bacillus subtilis and the concentration of Bacillus subtilis in the animal feed is between 1×10² and 1×10⁶ CFU/g of said animal feed.
